(19) United States
(12) Patent Application Publication
(10) Pub. No.: US 2024/0285349 A1
(43) Pub. Date: Aug. 29, 2024

(54) APPARATUS AND METHOD FOR THREE-DEMENSIONAL NAVIGATION OF MEDICAL TOOL

(71) Applicant: KOREA INSTITUTE OF SCIENCE AND TECHNOLOGY, Seoul (KR)

(72) Inventors: Jinseok KIM, Seoul (KR); Minsu JANG, Seoul (KR); Deukhee LEE, Seoul (KR); Hyehyun HAN, Seoul (KR); Byung Kook KIM, Seoul (KR); Byung Chul LEE, Seoul (KR)

(73) Assignee: KOREA INSTITUTE OF SCIENCE AND TECHNOLOGY, Seoul (KR)

(21) Appl. No.: 18/422,685

(22) Filed: Jan. 25, 2024

(30) Foreign Application Priority Data
Feb. 28, 2023 (KR) 10-2023-0027176

(51) Int. Cl. A61B 34/20; G06T 7/00; G06T 7/33; G06T 7/50; G06T 15/00; G16H 30/20

(52) U.S. Cl. CPC A61B 34/20; G06T 7/0014; G06T 7/337; G06T 7/50; G06T 15/00; G16H 30/20; A61B 2034/2065; G06T 2200/04; G06T 2207/30004

(57) ABSTRACT

Disclosed embodiments aim to provide an apparatus and method for three-dimensional navigation of a medical tool that enables identifying the three-dimensional shape and position of the medical tool within a human body. To achieve the above object, the disclosed embodiment includes a three-dimensional high-resolution image acquisition unit configured to acquire a three-dimensional high-resolution image of a human organ that is targeted for a medical procedure; a three-dimensional shape data acquisition unit configured to acquire three-dimensional shape data of a medical tool of a flexible material in real time; a registration unit configured to register the three-dimensional high-resolution image and the three-dimensional shape data with a reference point; and a display unit configured to display an image registered by the registration unit.

### DESCRIPTION OF GOVERNMENT-SPONSORED RESEARCH

This research was supported by the [Ministry of Science and ICT, Project No.: 1711196535, Subproject No.: 2E32340, Project Name: Research on Overcoming Disabilities in Response to the Super Aging Population].

This research was supported by the [Ministry of Science and ICT, Project Number: 1711174363, Subproject Number: KD000145, Project Name: Development of a three-dimensional navigation system with real-time human body deformation modeling applied].

This research was supported by the [Ministry of Science and ICT, Project No.: 1345359429, Subproject No.: 2021R1A6A3A01087549, Project Title: Development of Optical Fiber Sensing System for Real-time Three-dimensional Shape and Position Monitoring of Flexible Material Medical Tool].

### CROSS-REFERENCE TO RELATED APPLICATIONS

This application claims the benefit under 35 USC 119 (a) of Korean Patent Application No. 10-2023-0027176 filed on Feb. 28, 2023, in the Korean Intellectual Property Office, the entire disclosure of which is incorporated herein by reference for all purposes.

### BACKGROUND OF THE INVENTION

### Field of the Invention

Disclosed embodiments relate to an apparatus and method for three-dimensional navigation of a medical tool, and more particularly, to an apparatus and method for three-dimensional navigation of a medical tool that enables identifying the three-dimensional shape and position of the medical tool within a human body.

### Description of the Related Art

When a medical tool of flexible material is inserted into a bent human organ (e.g., blood vessel, digestive system, prostate, etc.) for a medical procedure such as an endoscopic procedure or a catheter procedure, the medical tool is conventionally inserted into the human organ by relying only on a tactile sensation of a physician's hand to perform the medical procedure.

This causes a conventional problem of high potential of an occurrence of medical accidents.

To solve this problem, in the related art, the shape and position of the medical tool is measured in real time by X-ray imaging.

However, there has been a problem in that measuring the shape and position of the medical tool by X-ray imaging can only identify the shape and position of the medical tool in a two-dimensional view, and there is a high risk of radiation exposure.

### SUMMARY OF THE INVENTION

Disclosed embodiments are directed to solving the conventional problems as described above, and an object of the disclosed embodiments is to provide an apparatus and method for three-dimensional navigation of a medical tool that enables identifying the three-dimensional shape and position of a medical tool in a human body by providing an image acquired by registering a three-dimensional high-resolution image and three-dimensional shape data with a reference point when the three-dimensional shape data of the medical tool is acquired through a shape sensor equipped on the medical tool after the three-dimensional high-resolution image of a human organ that is targeted for a medical procedure is acquired.

Another object of the disclosed embodiments is to provide an apparatus and method for three-dimensional navigation of a medical tool that enables identifying the three-dimensional shape and position of a medical tool in a human body by providing an image acquired by registering a three-dimensional high-resolution image and three-dimensional shape data with a reference point when the three-dimensional shape data of the medical tool is acquired through a three-dimensional ultrasound device after the three-dimensional high-resolution image of a human organ that is targeted for a medical procedure is acquired.

There is provided an apparatus for three-dimensional navigation of a medical tool according to an embodiment in order to achieve the objects described above, the apparatus may include: a three-dimensional high-resolution image acquisition unit configured to acquire a three-dimensional high-resolution image of a human organ that is targeted for a medical procedure; a three-dimensional shape data acquisition unit configured to acquire three-dimensional shape data of a medical tool of a flexible material in real time; a registration unit configured to register the three-dimensional high-resolution image and the three-dimensional shape data with a reference point; and a display unit configured to display an image registered by the registration unit.

In addition, in the apparatus for three-dimensional navigation of a medical tool according to the disclosed embodiment, the three-dimensional high-resolution image acquisition unit may acquire the three-dimensional high-resolution image of the human organ, including a marker marked at a point at which the medical tool is to be inserted, and the registration unit may determine the marker included in the three-dimensional high-resolution image as the reference point and register the three-dimensional high-resolution image and the three-dimensional shape data with the reference point as a start point of the three-dimensional shape data.

In addition, in the apparatus for three-dimensional navigation of a medical tool according to the disclosed embodiment, the marker may mark a point at which the medical tool is to be inserted and be a reference for the x, y, and z axes forming a three-dimensional coordinate system on the three-dimensional high-resolution image.

In addition, in the apparatus for three-dimensional navigation of a medical tool according to the disclosed embodiment, the registration unit may determine a portion having a specific shape among shapes of the human organ as the reference point and register the three-dimensional high-resolution image and the three-dimensional shape data by matching the determined reference point in a three-dimensional coordinate system, and in which the three-dimensional high-resolution image and the three-dimensional shape data may be registered by matching the portion having the specific shape among the shapes of the human organ and a portion having a specific shape among shapes of the medical tool in the three-dimensional coordinate system.

In addition, in the apparatus for three-dimensional navigation of a medical tool according to the disclosed embodiment, the apparatus may further include a tube of a specific shape positioned a point in which the medical tool is inserted, in which the three-dimensional high-resolution image acquisition unit may acquire the three-dimensional high-resolution image of the human organ, including the tube of the specific shape, and in which the registration unit may determine the tube of the specific shape included in the three-dimensional high-resolution image as the reference point and register the three-dimensional high-resolution image and the three-dimensional shape data with the reference point as a start point of the three-dimensional shape data.

In addition, in the apparatus for three-dimensional navigation of a medical tool according to the disclosed embodiment, the registration unit, when registering the three-dimensional high-resolution image and the three-dimensional shape data with the reference point, may predict, through physical properties of the human organ, a change in a shape of the organ in a portion of the human organ in which the medical tool is inserted after the medical tool is inserted, and convert the three-dimensional high-resolution image based on the predicted change in the shape of the organ.

In addition, in the apparatus for three-dimensional navigation of a medical tool according to the disclosed embodiment, the registration unit, when the three-dimensional shape data acquisition unit acquires the three-dimensional shape data of the medical tool through an external device, may extract three-dimensional shape information capable of identifying a shape of the human organ from the three-dimensional image acquired through the external device, and convert the three-dimensional high-resolution image based on the extracted three-dimensional shape information.

In addition, in the apparatus for three-dimensional navigation of a medical tool according to the disclosed embodiment, the registration unit may register the three-dimensional high-resolution image and the three-dimensional shape data with the reference point, and then identify whether a non-matching portion exists in the registered three-dimensional high-resolution image and the three-dimensional shape data, and when the non-matching portion exists, convert the three-dimensional high-resolution image of the non-matching portion based on the three-dimensional shape data.

In addition, in the apparatus for three-dimensional navigation of a medical tool according to the disclosed embodiment, the three-dimensional shape data acquisition unit may acquire three-dimensional shape data of the medical tool through a shape sensor equipped on the medical tool of a flexible material.

In addition, in the apparatus for three-dimensional navigation of a medical tool according to the disclosed embodiment, the three-dimensional shape data acquisition unit may acquire three-dimensional shape data of the medical tool through an external device.

Further, there is provided a method of three-dimensional navigation of a medical tool according to an embodiment in order to achieve the objects described above, the method may include: acquiring a three-dimensional high-resolution image of a human organ that is targeted for a medical procedure in a three-dimensional high-resolution image acquisition unit; acquiring three-dimensional shape data of the medical tool of a flexible material in real time in a three-dimensional shape data acquisition unit; registering the three-dimensional high-resolution image and the three-dimensional shape data with a reference point in a registration unit; and displaying the registered image in a display unit.

In addition, in the method of three-dimensional navigation of a medical tool according to the disclosed embodiment, the acquiring of the three-dimensional high-resolution image may be acquiring the three-dimensional high-resolution image of the human organ, including a marker marked at a point at which the medical tool is to be inserted, and in which the registering of the three-dimensional high-resolution image and the three-dimensional shape data with the reference point may be determining the marker included in the three-dimensional high-resolution image as the reference point and registering the three-dimensional high-resolution image and the three-dimensional shape data with the reference point as a start point of the three-dimensional shape data.

In addition, in the method of three-dimensional navigation of a medical tool according to the disclosed embodiment, the registering of the three-dimensional high-resolution image and the three-dimensional shape data with the reference point may be determining a portion having a specific shape among shapes of the human organ as the reference point and registering the three-dimensional high-resolution image and the three-dimensional shape data by matching the determined reference point in a three-dimensional coordinate system, and in which the three-dimensional high-resolution image and the three-dimensional shape data may be registered by matching the portion having the specific shape among the shapes of the human organ and a portion having a specific shape among shapes of the medical tool in the three-dimensional coordinate system.

In addition, in the method of three-dimensional navigation of a medical tool according to the disclosed embodiment, the acquiring of the three-dimensional high-resolution image may be acquiring the three-dimensional high-resolution image of the human organ, including a tube of a specific shape positioned at a point at which the medical tool is to be inserted, and in which the registering of the three-dimensional high-resolution image and the three-dimensional shape data with the reference point may be determining the tube of the specific shape included in the three-dimensional high-resolution image as a reference point and registering the three-dimensional high-resolution image and the three-dimensional shape data with the reference point as a start point of the three-dimensional shape data.

In addition, in the method of three-dimensional navigation of a medical tool according to the disclosed embodiment, the registering of the three-dimensional high-resolution image and the three-dimensional shape data with the reference point, when the three-dimensional high-resolution image and the three-dimensional shape data are registered with the reference point, may be predicting, through physical properties of the human organ, a change in a shape of the organ in a portion of the human organ in which the medical tool is inserted after the medical tool is inserted, and converting the three-dimensional high-resolution image based on the predicted change in the shape of the organ.

In addition, in the method of three-dimensional navigation of a medical tool according to the disclosed embodiment, the registering of the three-dimensional high-resolution image and the three-dimensional shape data with the reference point, when the three-dimensional shape data of the medical tool are acquired through an external device in the acquiring of the three-dimensional shape data, may be extracting three-dimensional shape information capable of identifying a shape of the human organ from the three-dimensional image acquired through the external device and converting the three-dimensional high-resolution image based on the extracted three-dimensional shape information.

In addition, in the method of three-dimensional navigation of a medical tool according to the disclosed embodiment, the registering of the three-dimensional high-resolution image and the three-dimensional shape data with the reference point may be registering the three-dimensional high-resolution image and the three-dimensional shape data with the reference point, then identifying whether a non-matching portion exists in the registered three-dimensional high-resolution image and the three-dimensional shape data, and when the non-matching portion exists, converting the three-dimensional high-resolution image of the non-matching portion based on the three-dimensional shape data.

In addition, in the method of three-dimensional navigation of a medical tool according to the disclosed embodiment, the acquiring of the three-dimensional shape data of the medical tool of a flexible material may be acquiring the three-dimensional shape data of the medical tool through a shape sensor equipped on the medical tool of a flexible material.

In addition, in the method of three-dimensional navigation of a medical tool according to the disclosed embodiment, the acquiring of the three-dimensional shape data of the medical tool of a flexible material may be acquiring the three-dimensional shape data of the medical tool through an external device.

Other specific details of the embodiments are included in the “detailed description of the invention” and the “drawings” attached hereto.

Advantages and features of the disclosed embodiments and methods of achieving the advantages and features will be clear with reference to various embodiments described in detail below together with the accompanying drawings.

However, it should be understood that the disclosed embodiments are not limited to the configuration of each of the embodiments disclosed below, but may also be implemented in a variety of other forms, and that each of the embodiments disclosed herein is provided only to make the disclosure of the disclosed embodiments complete and to fully inform those skilled in the art to which the disclosed embodiments belong of the scope of the disclosed embodiments, and that the disclosed embodiments are only defined by the scope of each claim of the appended claims.

According to the disclosed embodiments, it may be provided to identify the three-dimensional shape and position of the medical tool of a flexible material in the human body during a medical procedure.

### BRIEF DESCRIPTION OF THE DRAWINGS

FIG. 1 is a schematic view illustrating a configuration of an apparatus for three-dimensional navigation of a medical tool, according to an embodiment.

FIG. 2 is a view exemplarily illustrating a three-dimensional high-resolution image of a human organ obtained, according to an embodiment.

FIGS. 3 and 4 are views exemplarily illustrating a shape sensor according to an embodiment.

FIG. 5 is a view exemplarily illustrating a coupled state of the medical tool and the shape sensor, according to an embodiment.

FIG. 6 is a view exemplarily illustrating a three-dimensional ultrasound device according to an embodiment.

FIGS. 7A and 7B are a view exemplarily illustrating a registration state of a three-dimensional high-resolution image and three-dimensional shape data being registered through a reference point, according to an embodiment.

FIG. 8 is a view exemplarily illustrating a display state of a marker that is applied in an embodiment.

FIG. 9 is a view exemplarily illustrating a state in which a tube of a specific shape is equipped, which is applied in an embodiment.

FIG. 10 is a view exemplarily illustrating a change in shape of a human organ predicted, according to an embodiment.

FIGS. 11A to 11D are a view exemplarily illustrating three-dimensional high-resolution images and three-dimensional shape data being matched, according to an embodiment.

FIG. 12 is a view exemplarily illustrating three-dimensional high-resolution images and three-dimensional shape data being matched, according to an embodiment.

FIG. 13 is a processing flowchart for describing a method of three-dimensional navigation of the medical tool, according to an embodiment.

### DETAILED DESCRIPTION OF THE INVENTION

It should be understood that before describing the disclosed embodiments in detail, the terms and words used in the present specification are not to be interpreted unconditionally and without limitation in the general or dictionary meaning, and that the inventor of the disclosed embodiments may appropriately define and use the concepts of various terms to best describe his/her own invention, and further that these terms and words are to be interpreted in a meaning and concept consistent with the technical spirit of the disclosed embodiments.

That is, it should be understood that the terms used in the present specification are used only to describe preferred examples of the disclosed embodiments and are not intended to specifically limit the content of the disclosed embodiments, and that these terms are terms defined in consideration of the various possibilities of the disclosed embodiments.

In addition, in the present specification, it should be understood that singular expressions may include plural expressions unless the context clearly indicates a different meaning, and similarly, the plural expressions may have a singular meaning.

Throughout the present specification, where a constituent element is described as “comprising/including” another element, which, unless specifically stated to the contrary, may mean to include any other constituent element and not to exclude any other constituent element.

Further, when a constituent element is described as “existing within, or being installed in connection with,” another constituent element, it should be understood that the constituent element may be directly connected to, installed in contact with, or installed spaced a certain distance apart from another constituent element, and that in case of being installed spaced a certain distance apart, there may be a third constituent element or means for fixing or connecting the constituent element to another constituent element, and the description of the third constituent element or means may be omitted.

In contrast, when a constituent element is described as being “directly connected” or “directly accessed” to another constituent element, it should be understood that there is no third constituent element or means.

Similarly, other expressions that describe the relationship between respective constituent elements, such as “between” and “directly between”, or “adjacent to” and “directly adjacent to”, should be interpreted in the same manner.

In addition, it should be understood that when the terms “one surface,” “the other surface,” “one side,” “the other side,” “first,” “second,” and the like, are used in the present specification, they are used to refer to one constituent element so that this one constituent element can be clearly distinguished from other constituent elements, and that the meaning of the corresponding constituent element is not limited by such terms.

In addition, when the terms relating to a position, such as “top,” “bottom,” “left,” “right,” and the like, are used in the present specification, it should be understood that they refer to a relative position in the corresponding drawing with respect to the corresponding constituent element, and should not be understood that the terms relating to a position refer to an absolute position, unless the absolute position is specified with respect to the constituent element.

Further, it should be understood that in the disclosed embodiments, the terms ““unit,”” “device,” “module,” “apparatus,” and the like, when used, mean a unit capable of performing one or more functions or operations, which may be implemented in hardware or software, or a combination of hardware and software.

In addition, in specifying the reference numeral for each constituent element in each drawing, the present specification is intended to indicate that the same constituent element has the same reference numeral even though it is illustrated in different drawings, i.e., the same reference numeral throughout the specification refers to the same constituent element.

In the drawings accompanying the present specification, the size, position, coupling relationships, etc. of each of the constituent elements constituting the disclosed embodiments may be exaggerated, reduced, or omitted in some respects in order to convey the spirit of the disclosed embodiments with sufficient clarity or for convenience of description, and thus the proportions or scales may not be strictly accurate.

In addition, in describing the disclosed embodiments below, detailed descriptions of the configuration, for example, of known art, including prior art, may be omitted where it is determined that such descriptions would unnecessarily obscure the subject matter of the disclosed embodiments.

Hereinafter, with reference to the accompanying drawings, an apparatus and method for three-dimensional navigation of a medical tool according to the disclosed embodiments will be described in detail.

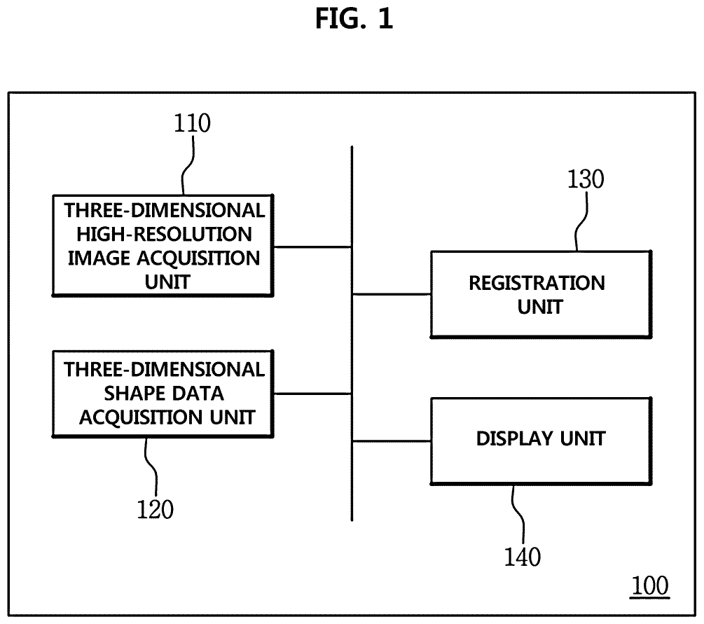

FIG. 1 is a schematic view illustrating a configuration of an apparatus for three-dimensional navigation of a medical tool, according to an embodiment.

As illustrated in FIG. 1, an apparatus 100 for three-dimensional navigation of a medical tool according to a disclosed embodiment may include a three-dimensional high-resolution image acquisition unit 110, a three-dimensional shape data acquisition unit 120, a registration unit 130, a display unit 140, and the like.

The three-dimensional high-resolution image acquisition unit 110 may acquire a three-dimensional high-resolution image of a human organ (e.g., blood vessels, digestive system, prostate, etc.) that is targeted for a medical procedure.

Here, the medical procedure may be, but is not limited to, a catheter procedure, an endoscopic procedure, and the like using a medical tool of a flexible material.

The three-dimensional high-resolution image acquisition unit 110 may acquire the three-dimensional high-resolution image of the human organ through computed tomography (CT), magnetic resonance imaging (MRI), C-shaped radiographic imaging equipment (C-arm), an ultrasound device, and the like, but is not limited thereto.

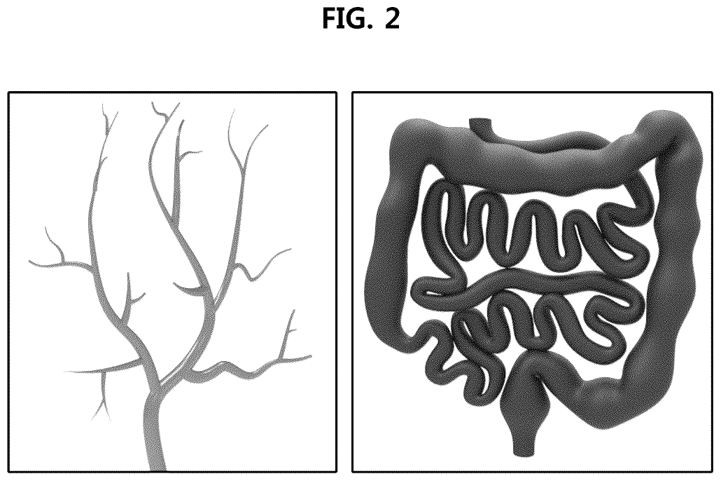

The three-dimensional high-resolution image acquisition unit 110 preferably acquires the three-dimensional high-resolution image of the human organ that is targeted for the medical procedure as illustrated in FIG. 2 through preoperative photographing.

The three-dimensional shape data acquisition unit 120 may acquire three-dimensional shape data of the medical tool of a flexible material in real time during the medical procedure.

The three-dimensional shape data acquisition unit 120 may acquire the three-dimensional shape data of the medical tool in real time through a shape sensor equipped on the medical tool of a flexible material.

While the embodiment of the present invention has been described with an example in which the shape sensor is equipped with the medical tool, the shape sensor may not be equipped with the medical tool.

In the disclosed embodiments, the shape sensor equipped on the medical tool of a flexible material may be implemented as a sensor capable of representing a three-dimensional shape, such as an optical fiber sensor, a magnetic field sensor, or a strain sensor, but is not limited thereto.

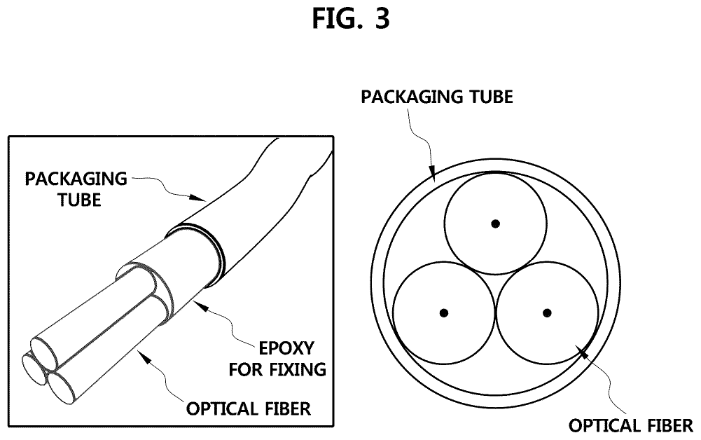

When the shape sensor is implemented based on the optical fiber sensor, the shape sensor may be implemented with three strands of optical fiber fixed in one tube to acquire three-dimensional shape data, as illustrated in FIG. 3. When curvature occurs in the shape sensor, each of the three strands of optical fiber is subjected to different strains, and a direction of curvature and magnitude of curvature may be calculated from the degree of stretching/extension. Here, the optical fiber sensor may use an optical fiber capable of strain measurement, such as a fiber Bragg grating (FBG), Brillouin, or long period grating (LPG).

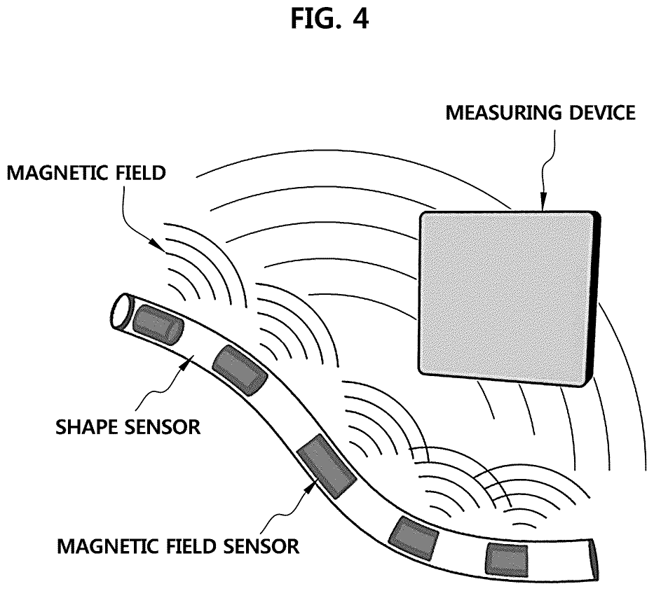

When the shape sensor is implemented based on a magnetic field sensor, as illustrated in FIG. 4, the magnetic field sensor may measure a distance from a measuring device externally installed through a magnetic field and display the distance in three-dimensional coordinates to implement a shape sensor capable of acquiring the three-dimensional shape data.

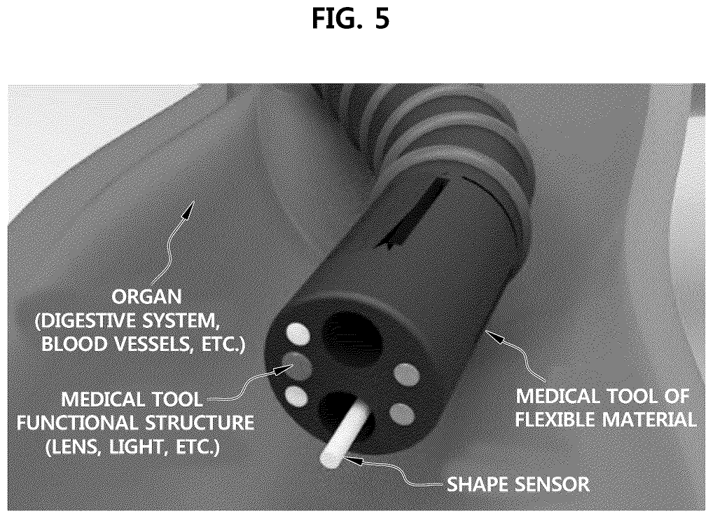

The aforementioned shape sensor may be equipped inside the medical tool of a flexible material, specifically in a manner such that the shape sensor is inserted into a tool channel of the medical tool, as illustrated in FIG. 5.

In addition, the shape sensor may be equipped in a manner to be attached to an exterior of the medical tool.

As described above, the shape sensor, which may be equipped inside or outside the medical tool, is preferably equipped to accurately follow the shape of the medical tool.

In addition, the three-dimensional shape data acquisition unit 120 may acquire the three-dimensional shape data of the medical tool in real time through a separate external device.

The external device that acquires the three-dimensional shape data of the medical tool in real time may be a three-dimensional ultrasound device, and the three-dimensional shape data of the medical tool may be acquired in real time through the three-dimensional ultrasound device.

When the three-dimensional shape data acquisition unit 120 acquires the three-dimensional shape data of the medical tool through the external device, such as a three-dimensional ultrasound device, the external device may be equipped with a sensor for measuring a three-dimensional movement distance and a direction or position of the external device in order to identify a position in a three-dimensional coordinate system of the three-dimensional shape data of the medical tool being measured by the external device.

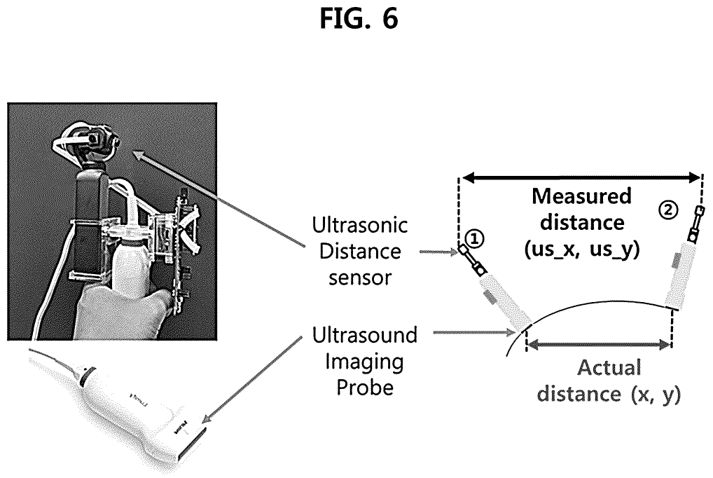

For example, as illustrated in FIG. 6, an ultrasonic distance measurement sensor may be equipped on an end of a probe constituting the three-dimensional ultrasound device to measure a time for an ultrasonic wave emitted by the sensor to bounce off a wall and return to identify a position in a three-dimensional space, which is then represented as a position in the three-dimensional coordinate system of the ultrasound probe, thereby identifying a movement distance and direction or position in the three-dimensional coordinate system of the ultrasound probe.

While the disclosed embodiment describes an ultrasonic distance measurement sensor as an example of a sensor for measuring the three-dimensional movement distance and direction or position of the external device, the present invention is not limited thereto, and other sensors such as an inertial measurement unit (IMU), an ultra-wide band (UWB), a marker-optical sensor may be used.

In addition, while the disclosed embodiment describes the three-dimensional ultrasound device as an example of the external device that acquires the three-dimensional shape data of the medical tool in real time, the present invention is not limited thereto, and the external device may be variously implemented as a device that is capable of acquiring the three-dimensional shape data of the medical tool in real time, such as a three-dimensional X-ray.

The three-dimensional shape data acquisition unit 120 may use the shape sensor and the external device separately to acquire the three-dimensional shape data of the medical tool, and may use the shape sensor and the external device together to further improve the accuracy of the acquired three-dimensional shape data.

Meanwhile, the registration unit 130 may register the three-dimensional high-resolution image acquired by the three-dimensional high-resolution image acquisition unit 110 and the three-dimensional shape data acquired by the three-dimensional shape data acquisition unit 120 with a reference point.

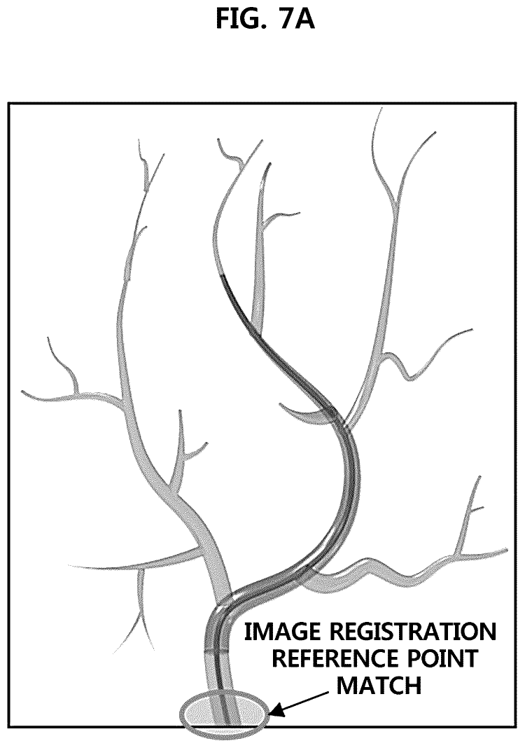

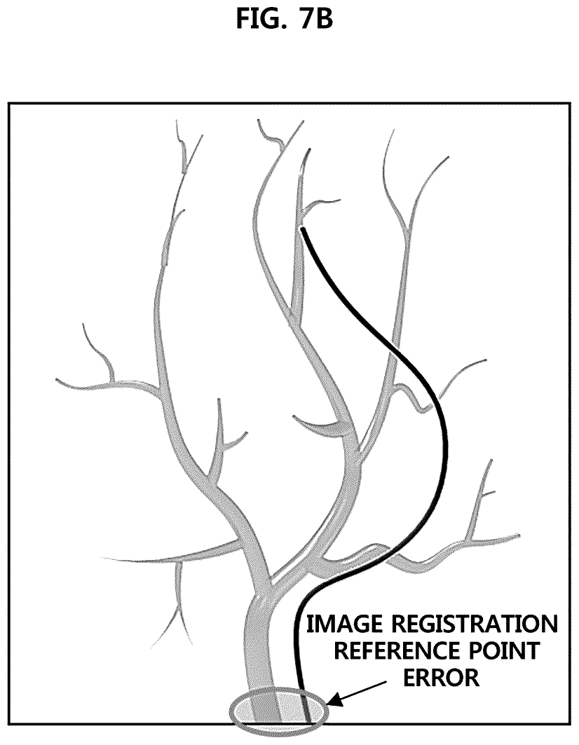

Here, the reference point is a point required to match a three-dimensional coordinate system of the three-dimensional high-resolution image with a three-dimensional coordinate system of the three-dimensional shape data to combine the three-dimensional high-resolution image and the three-dimensional shape data into one image, when the reference point is determined accurately, the medical tool and the human organ are matched, as illustrated in FIG. 7A, and when the reference point is not determined accurately, an error may occur, resulting in the medical tool being represented outside the human organ, that is, on a path that the medical tool cannot proceed, as illustrated in FIG. 7B.

As described above, the registration unit 130 registers the three-dimensional high-resolution image and the three-dimensional shape data with the reference point, and a method of determining the reference point may be as follows.

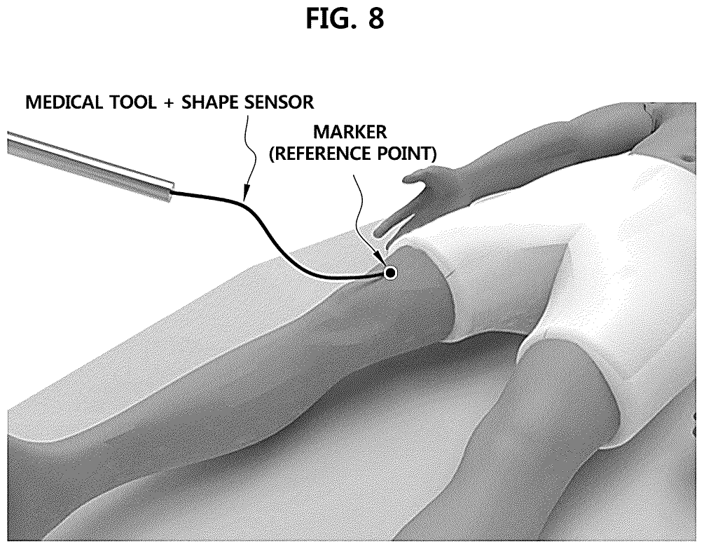

For example, a marker is marked at a point where the medical tool is to be inserted, as illustrated in FIG. 8, before the three-dimensional high-resolution image of the human organ that is targeted for the medical procedure is acquired through the three-dimensional high-resolution image acquisition unit 110. When the three-dimensional high-resolution image of the human organ is acquired through the three-dimensional high-resolution image acquisition unit 110, the three-dimensional high-resolution image of the human organ is acquired including the marker at the point where the medical tool is to be inserted, so that the marker may be included in the three-dimensional high-resolution image.

As described above, the marker marked at the point where the medical tool is to be inserted is preferably made of a material that may appear in a final image depending on a type of the three-dimensional high-resolution image acquisition unit 110, such as a metal, fluorescent, or contrast material.

As described above, when the three-dimensional high-resolution image acquisition unit 110 acquires the three-dimensional high-resolution image of the human organ including the marker marked at the point where the medical tool is to be inserted, the registration unit 130 determines the marker included in the three-dimensional high-resolution image as the reference point, and may register the three-dimensional high-resolution image and the three-dimensional shape data using the reference point as a start point for the three-dimensional shape data. Here, determining the marker as the reference point may be achieved by specifying a portion where the marker is positioned from an operator, or through an image recognition technology using artificial intelligence.

The markers included in the three-dimensional high-resolution image acquired by the three-dimensional high-resolution image acquisition unit 110 may indicate a point at which the medical tool is to be inserted, but may also serve as a reference for the x, y, and z axes that may form a three-dimensional coordinate system in the three-dimensional high-resolution image.

The registration unit 130 may represent in the three-dimensional coordinate system only a portion of the three-dimensional shape data of the medical tool acquired through the three-dimensional shape data acquisition unit 120 that is inserted into the human organ.

As another example, the registration unit 130 may determine a portion of the shape of the human organ in the three-dimensional high-resolution image acquired through the three-dimensional high-resolution image acquisition unit 110 that has a specific shape, such as, for example, a straight shape, an S-shape, or the like, as the reference point, and register the three-dimensional high-resolution image and the three-dimensional shape data by matching the determined reference point in the three-dimensional coordinate system.

Specifically, the three-dimensional high-resolution image and the three-dimensional shape data may be registered by determining a portion of the shape of the human organ in the three-dimensional high-resolution image acquired through the three-dimensional high-resolution image acquisition unit 110 that has a specific shape, such as, for example, a straight shape, an S-shape, or the like, as the reference point, and matching the portion determined as the reference point with a portion of the three-dimensional shape data of the medical tool acquired through the three-dimensional shape data acquisition unit 120 that has a specific shape.

Here, determining the portion of the shape of the human organ that has a specific shape as the reference point may be achieved by specifying a portion having the specific shape from an operator, or through an image recognition technology using artificial intelligence.

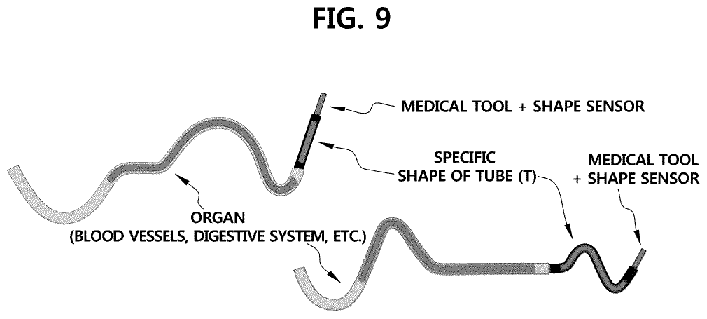

As another example, a tube T of a specific shape is equipped at a point where the medical tool is to be inserted, as illustrated in FIG. 9, before the three-dimensional high-resolution image of the human organ that is targeted for the medical procedure is acquired through the three-dimensional high-resolution image acquisition unit 110. When the three-dimensional high-resolution image of the human organ is acquired through the three-dimensional high-resolution image acquisition unit 110, the three-dimensional high-resolution image of the human organ is acquired including the tube T of a specific shape equipped at the point where the medical tool is to be inserted, so that the tube T of a specific shape may be included in the three-dimensional high-resolution image.

As described above, when the three-dimensional high-resolution image acquisition unit 110 acquires the three-dimensional high-resolution image of the human organ including the tube T of a specific shape equipped at the point where the medical tool is to be inserted, the registration unit 130 determines the tube T of a specific shape included in the three-dimensional high-resolution image as the reference point, and may register the three-dimensional high-resolution image and the three-dimensional shape data using the reference point as a start point for the three-dimensional shape data.

Here, determining the tube T of a specific shape as the reference point may be achieved by specifying a portion where the tube T of a specific shape is positioned from an operator, or through an image recognition technology using artificial intelligence.

Meanwhile, when the registration unit 130 registers the three-dimensional high-resolution image and the three-dimensional shape data with the reference point, the registration unit 130 may predict, through physical properties (stretchability, hardness, length, etc.) of the human organ, a change in a shape of the organ in a portion of the human organ in which the medical tool is inserted after the medical tool is inserted as the medical tool is inserted in the human organ and convert the three-dimensional high-resolution image based on the predicted change in the shape of the organ.

The registration unit 130 may predict a change in the shape of the organ after the medical tool is inserted in consideration of the properties of each human organ that is targeted for the medical procedure, that is, the physical properties, and then perform image registration based on the predicted change in the shape of the organ.

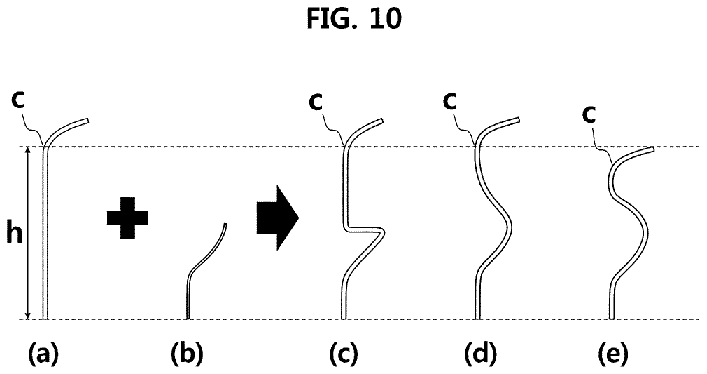

For example, as illustrated in FIG. 10, assuming that the human organ maintains a straight shape by a distance ‘h’ from the reference point and then experiences a bend C (see FIG. 10A), when the three-dimensional shape data acquired as the medical tool is inserted results in having a curvature from a straight shape (see FIG. 10B), it may be inferred that the shape of the human organ has changed as the medical tool is inserted.

In this case, it may be inferred that after the medical tool is inserted, the shape of the organ in the portion where the medical tool is inserted (a path that does not yet have three-dimensional shape data) may experience changes, which may be monitored by predicting the change in shape through the physical properties of the human organ.

In case that the shape of the human organ changes as the medical tool is inserted, unless the shape change of the portion where the medical tool is inserted after the medical tool is inserted (a path that does not yet have three-dimensional shape data) is predicted, a straight shape will be immediately obtained according to the pre-acquired three-dimensional high-resolution image, as illustrated in FIG. 10C, which may not happen unless the shape of the organ is fixed or experiencing extreme stretching.

Therefore, the registration unit 130 may predict a change in the shape of the human organ according to the insertion of the medical tool, but in consideration of the physical properties of the human organ that is targeted for the medical procedure, the registration unit 130 may predict the change in the shape of the organ in the portion where the medical tool is inserted after the medical tool is inserted.

For example, when the human organ has high stretchability, since the organ may be stretched by a distance distant to a distance ‘h’ depending on the physical properties of the stretchable human organ when the insertion of the medical tool results in curvature, the portion C where the bend occurs may be predicted to remain near ‘h’ as illustrated in FIG. 10D.

For example, when the human organ has low stretchability, since the organ is unlikely to be stretched depending on the physical properties of the low-stretchable human organ when the insertion of the medical tool results in curvature, the portion C where the curvature occurs may be predicted to occur at a shorter distance than ‘h’ as illustrated in FIG. 10E.

As described above, after the change in the shape of the organ is predicted in consideration of the physical properties of the human organ, the three-dimensional high-resolution image may be converted based on the predicted change in the shape of the organ.

The registration unit 130 described above may determine that the shape of the human organ has changed due to the insertion of the medical tool when, in the registration image of the three-dimensional high-resolution image and the three-dimensional shape data registered with the reference point, a tip portion of the medical tool in the three-dimensional shape data and the human organ in the three-dimensional high-resolution image do not match.

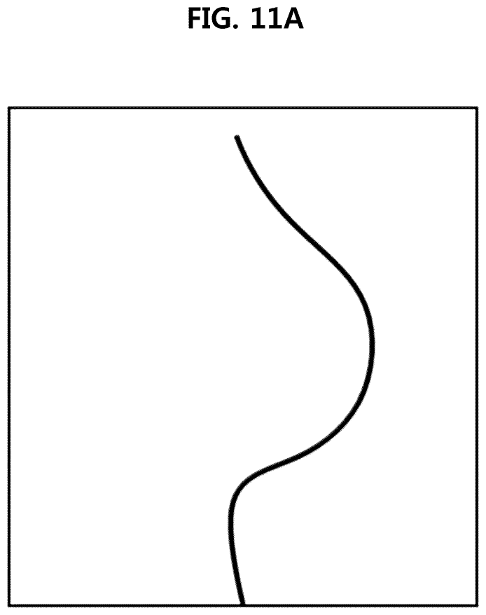

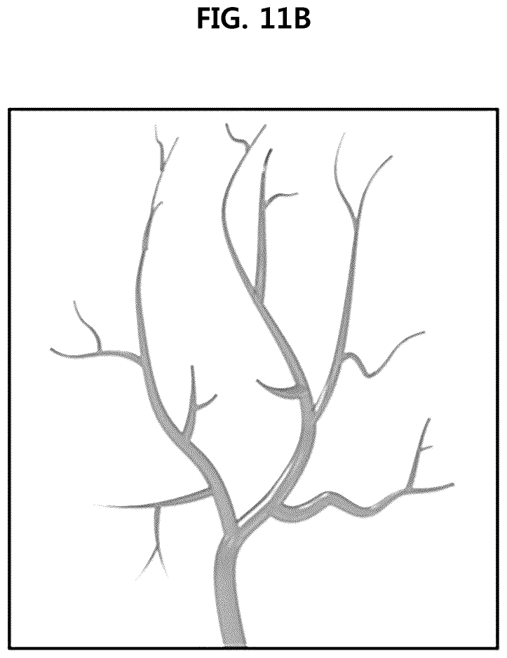

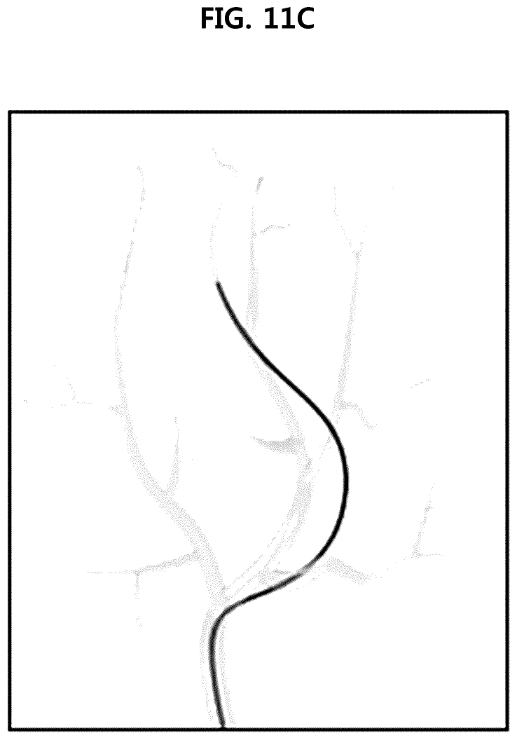

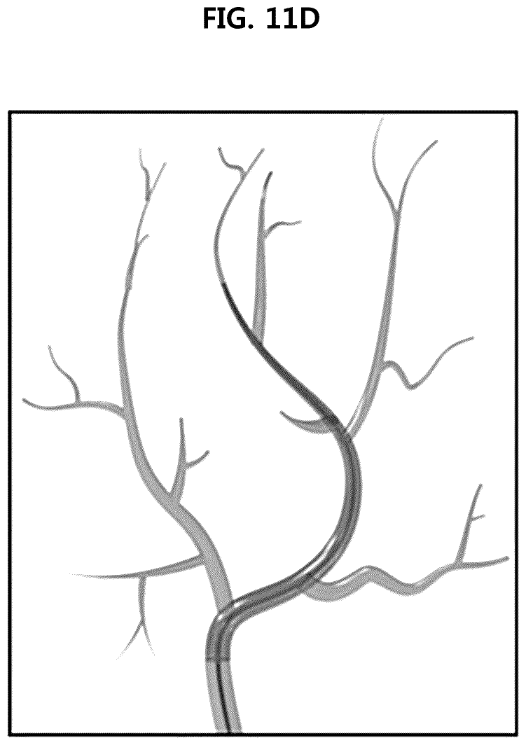

Meanwhile, the registration unit 130 may register the three-dimensional high-resolution image (FIG. 11B) and the three-dimensional shape data with the reference point (FIG. 11A), and then identify whether a non-matching portion exists in the registered three-dimensional high-resolution image and the three-dimensional shape data, and when the non-matching portion exists (FIG. 11C), the three-dimensional high-resolution image of the non-matching portion may be converted based on the three-dimensional shape data (FIG. 11D).

In addition, when the three-dimensional shape data acquisition unit 120 acquires the three-dimensional shape data of the medical tool through the external device such as the three-dimensional ultrasound device, the registration unit 130 may identify the three-dimensional shape of the human organ in the three-dimensional ultrasound image acquired using the external device such as the three-dimensional ultrasound device, and convert the three-dimensional high-resolution image based on the identified three-dimensional shape of the human organ.

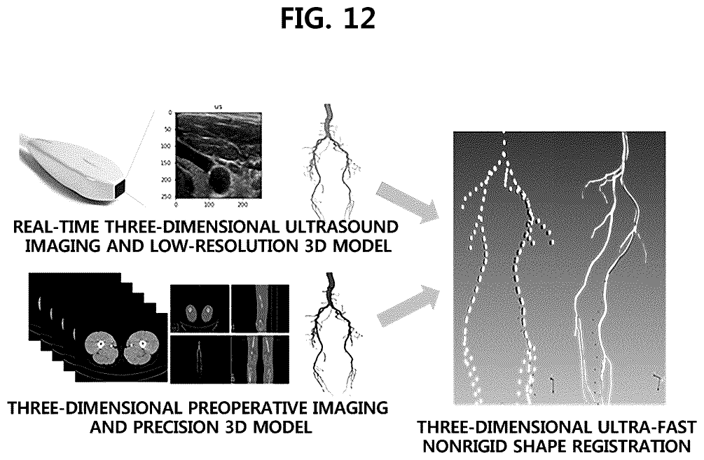

For example, as illustrated in FIG. 12, the three-dimensional ultrasound image acquired in real time using the external device, such as the three-dimensional ultrasound device, has a poor resolution, but three-dimensional shape information that enables identifying the shape of the human organ may be extracted. Here, the three-dimensional shape information extracted to identify the shape of the human organ may be a center point of a blood vessel, an outline of an organ, etc.

As described above, when the human organ shape information that enables identifying the shape of the human organ is extracted from the three-dimensional ultrasound image, the extracted human organ shape information is registered to the three-dimensional high-resolution image to identify the shape deformation of the human organ, that is, the non-matching portion, and when the shape deformation of the human organ is identified, the three-dimensional high-resolution image of the non-matching portion may be converted based on the shape information.

As mentioned above, when the three-dimensional shape data of the medical tool is acquired in real time using the external device such as the three-dimensional ultrasound device, the shape of the human organ may also be measured together, and thus, when the three-dimensional shape data of the medical tool and the three-dimensional high-resolution image are registered, the human organ shape information may be registered together.

Meanwhile, the display unit 140 may display an image registered by the registration unit 130.

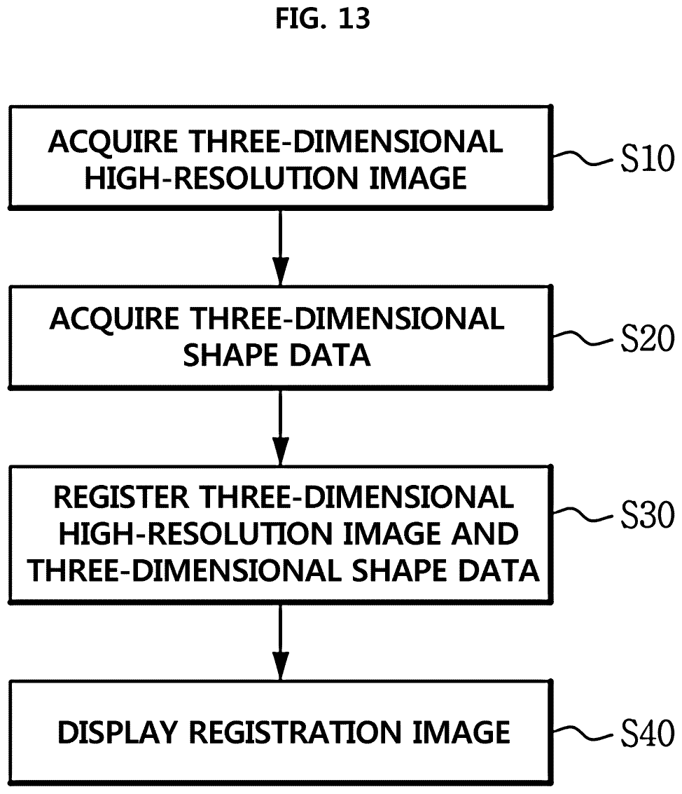

FIG. 13 is a processing flowchart for describing a method of three-dimensional navigation of the medical tool, according to an embodiment.

Since the method of three-dimensional navigation of a medical tool according to the disclosed embodiment proceeds on substantially the same configuration as the apparatus 100 for three-dimensional navigation of a medical tool illustrated in FIG. 1, the same reference numerals will be assigned to the same constituent elements as the apparatus 100 for three-dimensional navigation of a medical tool in FIG. 1, and repetitive descriptions will be omitted.

First, in step S10, the three-dimensional high-resolution image acquisition unit 110 may acquire a three-dimensional high-resolution image of the human organ that is targeted for the medical practice.

The three-dimensional high-resolution image acquisition unit 110 in step S10 described above acquires the three-dimensional high-resolution image of the human organ that is targeted for the medical procedure through preoperative photographing, but before acquiring the three-dimensional high-resolution image of the human organ that is targeted for the medical procedure, may acquire the three-dimensional high-resolution image of the human organ including a marker marked at a point where the medical tool is to be inserted, such that the three-dimensional high-resolution image includes the marker.

In addition, in step S10 described above, the three-dimensional high-resolution image acquisition unit 110 may, before acquiring the three-dimensional high-resolution image of the human organ that is targeted for the medical procedure, acquire the three-dimensional high-resolution image of the human organ by including a tube of a specific shape equipped at the point where the medical tool is to be inserted, such that the three-dimensional high-resolution image includes the tube of the specific shape.

Then, in step S20, the three-dimensional shape data acquisition unit 120 may acquire three-dimensional shape data of the medical tool of a flexible material in real time.

In step S20 described above, the three-dimensional shape data acquisition unit 120 may acquire the three-dimensional shape data of the medical tool in real time through a shape sensor equipped on the medical tool of a flexible material.

In addition, in step S20 described above, the three-dimensional shape data acquisition unit 120 may acquire the three-dimensional shape data of the medical tool in real time through a separate external device such as a three-dimensional ultrasound device.

In step S20 described above, the three-dimensional shape data acquisition unit 120 may use the shape sensor and the external device together to further improve the accuracy of the three-dimensional shape data of the medical tool.

Then, in step S30, the registration unit 130 may register the three-dimensional high-resolution image acquired through step S10 described above and the three-dimensional shape data acquired through step S20 described above with a reference point.

In step S30 described above, the registration unit 130 may determine the marker included in the three-dimensional high-resolution image acquired through step S10 described above as the reference point, and register the three-dimensional high-resolution image and the three-dimensional shape data using the reference point as a start point for the three-dimensional shape data.

In addition, in step S30 described above, the registration unit 130 may determine a portion of the shape of the human organ in the three-dimensional high-resolution image acquired through step S10 described above that has a specific shape, such as, for example, a straight shape, an S-shape, or the like, as the reference point, and register the three-dimensional high-resolution image and the three-dimensional shape data by matching the determined reference point in the three-dimensional coordinate system.

Specifically, in step S30 described above, the registration unit 130 may determine a portion of the shape of the human organ in the three-dimensional high-resolution image acquired through the three-dimensional high-resolution image acquisition unit 110 that has a specific shape, such as, for example, a straight shape, an S-shape, or the like, as the reference point, and match the portion determined as the reference point with a portion of the three-dimensional shape data of the medical tool acquired through the three-dimensional shape data acquisition unit 120 that has a specific shape in the three-dimensional coordinate system, thereby to register the three-dimensional high-resolution image and the three-dimensional shape data.

In addition, in step S30 described above, the registration unit 130 may determine the tube of a specific shape included in the three-dimensional high-resolution image acquired through step S10 described above as the reference point, and register the three-dimensional high-resolution image and the three-dimensional shape data using the reference point as a start point for the three-dimensional shape data.

In addition, in step S30 described above, when the three-dimensional shape data acquisition unit 120 acquires the three-dimensional shape data of the medical tool through the external device such as the three-dimensional ultrasound device in step S20 described above, a sensor capable of measuring movement and direction or position in the three-dimensional coordinate system is equipped on the external device so that a position in the three-dimensional coordinate system of the target being measured by the external device may be identified, and the registration unit 130 may register the three-dimensional high-resolution image and the three-dimensional shape data based on a position in the three-dimensional coordinate system of the medical tool being inserted into the human organ and measuring the human organ, using the position in the three-dimensional coordinate system of the external device measured from the sensor.

In addition, in step S30 described above, when registering the three-dimensional high-resolution image acquired in step S10 described above and the three-dimensional shape data acquired in step S20 described above with the reference point, the registration unit 130 may predict, through physical properties of the human organ, a change in a shape of the organ in a portion of the human organ in which the medical tool is inserted after the medical tool is inserted as the medical tool is inserted in the human organ and convert the three-dimensional high-resolution image based on the predicted change in the shape of the organ.

Also, in step S30 described above, when the three-dimensional shape data acquisition unit 120 acquires the three-dimensional shape data of the medical tool through the external device such as the three-dimensional ultrasound device in step S20 described above, the registration unit 130 may extract three-dimensional human organ shape information capable of identifying the shape of the human organ from a three-dimensional ultrasound image acquired through the external device such as the three-dimensional ultrasound device, register the extracted three-dimensional human organ shape information to the three-dimensional high-resolution image to identify shape deformation of the human organ, and, when the shape deformation of the human organ is identified, convert a three-dimensional high-resolution image of a portion in which the shape deformation is identified based on the human organ shape information.

When the three-dimensional shape data of the medical tool is acquired through the external device such as the three-dimensional ultrasound device, the three-dimensional shape data of the medical tool is also acquired along with human organ formation information. Therefore, the registration unit 130 may perform the conversion of the three-dimensional high-resolution image along with the registration of the three-dimensional shape data to the three-dimensional high-resolution image.

In addition, in step S30 described above, the registration unit 130 may register the three-dimensional high-resolution image acquired in step S10 described above and the three-dimensional shape data acquired in step S20 described above with the reference point, and then identify whether a non-matching portion exists in the registered three-dimensional high-resolution image and the three-dimensional shape data, and when the non-matching portion exists, convert the three-dimensional high-resolution image of the non-matching portion based on the three-dimensional shape data.

Then, in step S40, the display unit 140 may display the image registered through step S30 described above.

The method of three-dimensional navigation of a medical tool, according to the present invention, may be implemented as an application or in the form of program instructions that may be executed through various computer components and recorded on a computer-readable recording medium. The computer-readable recording medium may include program instructions, data files, data structures, or the like, in a stand-alone form or in a combination thereof.

The program instructions recorded in the computer-readable recoding medium may be designed and configured specifically for the disclosed embodiments or may be publicly known and available to those skilled in the field of computer software.

Examples of the computer-readable recording medium may include magnetic media, such as a hard disk, a floppy disk and a magnetic tape, optical media, such as CD-ROM and DVD, magneto-optical media, such as a floptical disk, and hardware devices, such as ROM, RAM and flash memory, which are specifically configured to store and run program instructions.

Examples of the program instructions include machine codes made by a compiler, as well as high-language codes that may be executed by a computer, using an interpreter.

As disclosed above, according to the disclosed embodiments, it is possible to identify the three-dimensional shape and position of the medical tool of a flexible material in the human body during the medical procedure, thereby reducing the possibility of occurrence of medical accidents, improving the accuracy of the medical procedure, and reducing the time of the medical procedure.

While the description above describes various preferred embodiments with some examples, it should be understood that the description of the various embodiments described in this “detailed description of the invention” section is merely illustrative, and those skilled in the art to which the disclosed embodiments belong can modify the disclosed embodiments from the above description to perform various other embodiments, or to perform embodiments equivalent to the disclosed embodiments. In addition, it should be understood that the disclosed embodiments are not limited by the description above, as the disclosed embodiments may be implemented in a variety of other forms, and that the above description is provided only to make the disclosure of the disclosed embodiments complete and to inform those skilled in the art to which the disclosed embodiments belong of the scope of the disclosed embodiments, and that the disclosed embodiments are only defined by the respective claims of the claims.

### DESCRIPTION OF REFERENCE NUMERALS

100. Apparatus for three-dimensional navigation of medical tool,
110. Three-dimensional high-resolution image acquisition unit,
120. Three-dimensional shape data acquisition unit,
130. Registration unit,
140. Display unit

What is claimed is:

1. An apparatus for three-dimensional navigation of a medical tool, the apparatus comprising:
one or more processors; and
a memory configured to store instructions executed by the one or more processors,
wherein the one or more processors are configured to:
acquire a three-dimensional high-resolution image of a human organ that is targeted for medical procedure;
acquire three-dimensional shape data of the medical tool of a flexible material in real time;
register the three-dimensional high-resolution image and the three-dimensional shape data with a reference point; and
instruct such that the registered image is displayed.

2. The apparatus of claim 1, wherein the one or more processors are configured to:
obtain the three-dimensional high-resolution image of the human organ, including a marker marked at a point at which the medical tool is to be inserted;
determine the marker included in the three-dimensional high-resolution image as a reference point; and
register the three-dimensional high-resolution image and the three-dimensional shape data with the reference point as a start point of the three-dimensional shape data.

3. The apparatus of claim 2, wherein the marker marks a point at which the medical tool is to be inserted and is a reference for the x, y, and z axes forming a three-dimensional coordinate system on the three-dimensional high-resolution image.

4. The apparatus of claim 1, wherein the one or more processors are configured to:
determine a portion having a specific shape among shapes of the human organ as the reference point; and
register the three-dimensional high-resolution image and the three-dimensional shape data by matching the reference point in a three-dimensional coordinate system, and
wherein the three-dimensional high-resolution image and the three-dimensional shape data are registered by matching the portion having the specific shape among the shapes of the human organ and a portion having a specific shape among shapes of the medical tool in the three-dimensional coordinate system.

5. The apparatus of claim 1, wherein the one or more processors are configured to:
acquire the three-dimensional high-resolution image of the human organ, including a tube of a specific shape positioned at a point at which the medical tool is to be inserted; determine the tube of the specific shape included in the three-dimensional high-resolution image as the reference point; and
register the three-dimensional high-resolution image and the three-dimensional shape data with the reference point as a start point of the three-dimensional shape data.

6. The apparatus of claim 1, wherein the one or more processors, when registering the three-dimensional high-resolution image and the three-dimensional shape data with the reference point, predict, through physical properties of the human organ, a change in a shape of the organ in a portion of the human organ in which the medical tool is inserted after the medical tool is inserted, and convert the three-dimensional high-resolution image based on the predicted change in the shape of the organ.

7. The apparatus of claim 1, wherein the one or more processors are configured to, when acquiring the three-dimensional shape data of the medical tool through an external device, extract three-dimensional shape information capable of identifying a shape of the human organ from the three-dimensional image acquired through the external device, and convert the three-dimensional high-resolution image based on the extracted three-dimensional shape information.

8. The apparatus of claim 1, wherein the one or more processors configured to register the three-dimensional high-resolution image and the three-dimensional shape data with the reference point, and then identify whether a non-matching portion exists in the registered three-dimensional high-resolution image and the three-dimensional shape data, and when the non-matching portion exists, convert the three-dimensional high-resolution image of the non-matching portion based on the three-dimensional shape data.

9. The apparatus of claim 1, wherein the one or more processors are configured to acquire the three-dimensional shape data of the medical tool through a shape sensor equipped on the medical tool of a flexible material.

10. The apparatus of claim 1, wherein the one or more processors are configured to acquire the three-dimensional shape data of the medical tool through an external device.

11. A method of three-dimensional navigation of a medical tool performed by a computing device including one or more processors, and a memory configured to store instructions executed by the one or more processors, the method comprising:
acquiring a three-dimensional high-resolution image of a human organ that is targeted for a medical procedure;
acquiring three-dimensional shape data of the medical tool of a flexible material in real time;
registering the three-dimensional high-resolution image and the three-dimensional shape data with a reference point; and
displaying the registered image.

12. The method of claim 11, wherein the acquiring of the three-dimensional high-resolution image is acquiring the three-dimensional high-resolution image of the human organ, including a marker marked at a point at which the medical tool is to be inserted, and
wherein the registering of the three-dimensional high-resolution image and the three-dimensional shape data with the reference point is determining the marker included in the three-dimensional high-resolution image as the reference point and registering the three-dimensional high-resolution image and the three-dimensional shape data with the reference point as a start point of the three-dimensional shape data.

13. The method of claim 11, wherein the registering of the three-dimensional high-resolution image and the three-dimensional shape data with the reference point is determining a portion having a specific shape among shapes of the human organ as the reference point and registering the three-dimensional high-resolution image and the three- dimensional shape data by matching the reference point in a three-dimensional coordinate system, and
wherein the three-dimensional high-resolution image and the three-dimensional shape data are registered by matching the portion having the specific shape among the shapes of the human organ and a portion having a specific shape among shapes of the medical tool in the three-dimensional coordinate system.

14. The method of claim 11, wherein the acquiring of the three-dimensional high-resolution image is acquiring the three-dimensional high-resolution image of the human organ, including a tube of a specific shape positioned at a point at which the medical tool is to be inserted, and
wherein the registering of the three-dimensional high-resolution image and the three-dimensional shape data with the reference point is determining the tube of the specific shape included in the three-dimensional high-resolution image as a reference point and registering the three-dimensional high-resolution image and the three-dimensional shape data with the reference point as a start point of the three-dimensional shape data.

15. The method of claim 11, wherein the registering of the three-dimensional high-resolution image and the three-dimensional shape data with the reference point is, when the three-dimensional high-resolution image and the three-dimensional shape data are registered with the reference point, predicting, through physical properties of the human organ, a change in a shape of the organ in a portion of the human organ in which the medical tool is inserted after the medical tool is inserted and converting the three-dimensional high-resolution image based on the predicted change in the shape of the organ.

16. The method of claim 11, wherein the registering of the three-dimensional high-resolution image and the three-dimensional shape data with the reference point is, when the three-dimensional shape data of the medical tool are acquired through an external device in the acquiring of the three-dimensional shape data, extracting three-dimensional shape information capable of identifying a shape of the human organ from the three-dimensional image acquired through the external device and converting the three-dimensional high-resolution image based on the extracted three-dimensional shape information.

17. The method of claim 11, wherein the registering of the three-dimensional high-resolution image and the three-dimensional shape data with the reference point is registering the three-dimensional high-resolution image and the three-dimensional shape data with the reference point, then identifying whether a non-matching portion exists in the registered three-dimensional high-resolution image and the three-dimensional shape data, and when the non-matching portion exists, converting the three-dimensional high-resolution image of the non-matching portion based on the three-dimensional shape data.

18. The method of claim 11, wherein the acquiring of the three-dimensional shape data of the medical tool of a flexible material is acquiring the three-dimensional shape data of the medical tool through a shape sensor equipped on the medical tool of a flexible material.

19. The method of claim 11, wherein the acquiring of the three-dimensional shape data of the medical tool of a flexible material is acquiring the three-dimensional shape data of the medical tool through an external device.

20. A non-transitory computer-readable recording medium configured to record a computer program for performing the method of three-dimensional navigation of a medical tool according to claim 11.
